(19) United States
(12) Patent Application Publication
(10) Pub. No.: US 2021/0268017 A1
(43) Pub. Date: Sep. 2, 2021

(54) MOLECULAR HYDROGEN-CONTAINING COMPOSITION FOR PREVENTION AND/OR IMPROVEMENT OF PNEUMONIA

(71) Applicant: MiZ Company Limited, Kamakura-shi (JP)

(72) Inventors: Fumitake SATOH, Kanagawa (JP); Bunpei Sato, Kanagawa (JP); Yusuke Ichikawa, Kanagawa (JP); Shinichi Hirano, Kanagawa (JP); Ryosuke Kurokawa, Kanagawa (JP)

(73) Assignee: MiZ Company Limited, Kamakura-shi (JP)

(21) Appl. No.: 17/185,450

(22) Filed: Feb. 25, 2021

(30) Foreign Application Priority Data
Feb. 28, 2020 (JP) 2020-046965

(51) Int. Cl. A61K 33/00; A61M 16/12; A61K 9/00; A61P 11/00

(52) U.S. Cl. CPC A61K 33/00; A61M 16/12; A61M 2202/02; A61P 11/00; A61K 9/007

(57) ABSTRACT

The present invention provides a composition for prevention and/or improvement of pneumonia. More specifically, the present invention provides a composition for prevention and/or improvement of pneumonia and a symptom associated with pneumonia in a subject, comprising molecular hydrogen as an active ingredient.

### RELATED APPLICATIONS

This application claims priority to Japanese Patent Application No. 2020-046965, filed on Feb. 28, 2020, the entire content of which is incorporated herein by reference.

### BACKGROUND OF THE INVENTION

### 1. Field of the Invention

The present invention provides a molecular hydrogen-containing composition for prevention and/or improvement of pneumonia in subjects.

### 2. Description of the Related Art

Pneumonia is a collective term for inflammatory diseases in the lungs. Each year, pneumonia develops in 450 million people worldwide (7% of the population), of whom 4 million patients died. In particular, since the first outbreak in Wuhan, China, the novel coronavirus (SARS-CoV-2) infection has spread all over the world from the end of 2019 to 2020 and has become a social problem as a global pandemic. As of Feb. 26, 2020, 81,006 patients with SARS-CoV-2 infection were identified worldwide, and of these, 2,764 patients died of respiratory diseases such as pneumonia caused by SARS-CoV-2 infection. However, no effective treatment methods have been found.

Hydrogen, the active ingredient of the present invention, has anti-oxidative reactivity, which reduces oxidative stress caused by reactive oxygen species, and a study in mice previously showed that hydrogen had improved chronic bronchitis and obstructive lung bronchitis (Japanese Patent No. 6628449). However, there are no precedent of the effect of hydrogen to improve refractory pneumonia having been documented so far.

Prevention and/or improvement of pneumonia would make it possible not only to achieve relief of distress and improvement in quality of life for patients, but also to contribute to reduction in healthcare costs. As described above, few components or substances are known to be useful for prevention and/or improvement of pneumonia.

Under such circumstances, an object of the present invention is to prevent and/or improve pneumonia by using molecular hydrogen and/or to promote improvement of a symptom associated with pneumonia.

### SUMMARY

That is, the present invention encompasses the following characteristics:

(1) A composition for prevention and/or improvement of pneumonia and a symptom associated with pneumonia in a subject, comprising administering to the subject a composition comprising an effective amount of molecular hydrogen.

(2) The composition according to (1), wherein the pneumonia is one or more pneumonia selected from the group consisting of bacterial pneumonia, viral pneumonia, fungal pneumonia, radiation pneumonia, and drug-induced pneumonia.

(3) The composition according to (1) or (2), wherein the symptom associated with pneumonia is one or more symptoms selected from the group consisting of sore throat, pyrexia, coughing, sneezing, sputum production, dyspnea, systemic malaise, nausea, fatigue, chest pain, shortness of breath, rhinorrhea, decreases in white blood cells, diarrhea, headache, reduced lung function, reduced heart function, and reduced kidney function.

(4) The composition according to any one of (1) to (3), wherein the composition is a liquid composition or a gas composition comprising the molecular hydrogen.

(5) The composition according to (5), wherein the liquid comprising the molecular hydrogen has a hydrogen concentration of 1 to 10 ppm.

(6) The composition according to (4), wherein the gas comprising the molecular hydrogen has a hydrogen concentration of higher than zero (0) and not higher than 18.5% by volume.

(7) The composition according to any one of (1) to (6), wherein the subject is a mammalian subject, including a human.

(8) The composition according to any one of (1) to (7), wherein the composition is produced by using a hydrogen gas generating apparatus, a hydrogen water generating apparatus, or a hydrogen gas adding apparatus.

The present invention can prevent and/or improve pneumonia in subjects. Additionally, refractory sepsis can also be improved by administering the composition according to the present invention to a subject.

### BRIEF DESCRIPTION OF THE DRAWINGS

FIG. 1 shows CT photographs taken before and after administration of hydrogen to a patient with interstitial pneumonia in Example 1; and

FIG. 2 shows CT photographs taken before and after administration of hydrogen to the patient with interstitial pneumonia in Example 1, which were taken at a site different from FIG. 1.

### DETAILED DESCRIPTION OF THE PREFERRED EMBODIMENTS

The present invention will be described in more detail below.

1. Composition for Prevention and/or Improvement of Pneumonia

The present invention provides a composition comprising molecular hydrogen as an active ingredient which promotes prevention and/or improvement of pneumonia and a symptom associated with pneumonia.

In the present specification, the term “pneumonia” is a collective term for inflammatory diseases of the lungs (alveoli, interstitium), excluding bronchitis.

In the present specification, “infectious pneumonia” is classified into bacterial pneumonia, viral pneumonia, and fungal pneumonia. Generally, secondary pneumonia following common cold or upper respiratory tract inflammation is bacterial pneumonia, but may be occasionally pneumonia or interstitial pneumonia caused by virus itself, such as influenza virus pneumonia, coronavirus pneumonia, and measles pneumonia. Noninfectious pneumonia, which is not caused by a pathogen, includes allergic hypersensitivity pneumonia.

In the present specification, the term “bacterial pneumonia” refers to acute inflammation of the lungs (pneumonia) caused by bacterial infection, and most cases thereof are alveolar pneumonia.

In the present specification, the term “bacterium causing bacterial pneumonia” refers to a Gram-positive bacterium, a Gram-negative bacterium, an anaerobic bacterium, an atypical bacterium, an acid-fast bacterium, mycoplasma, or the like.

In the present specification, the term “viral pneumonia” refers to acute inflammation of the lungs (pneumonia) caused by viral infection.

In the present specification, the term “causative virus of viral pneumonia” generally refers to rhinovirus, coronavirus, influenza virus, RS virus, adenovirus, parainfluenza virus, simple herpes virus, cytomegalovirus, or the like. Additionally, the term includes novel viruses having a mutation in the genome sequences of these viruses.

In the present specification, the term “fungal pneumonia” refers to acute inflammation of the lungs (pneumonia) caused by fungal infection.

In the present specification, the term “fungus” refers to Histoplasma capsulatum, Blastomyces, Cryptococcus, Pneumocystis, Coccidioides, or the like.

In the present specification, the term “drug-induced pneumonia” refers to an unexpected adverse reaction in the lungs caused by drip infusion or oral administration of the drug which is not the original effect of the drug. All drugs (in particular, antineoplastic drugs, interferons, anti-rheumatic drugs, biologics, and molecular target drugs), nutritional foods, and supplements have a potential to cause pulmonary disorders.

In the present specification, the term “radiation pneumonia” refers to a pulmonary disorder caused by radiation to develop, specifically pneumonia caused by radiation injury that occurs during radiation therapy against cancer that has developed in the chest such as lung cancer, esophagus cancer, and breast cancer, or the abdomen close to the lungs.

In the present specification, the term “refractory pneumonia” refers to pneumonia which is impossible or difficult to improve or resolve with current medicine or any drugs manufactured and marketed in the pharmaceutical industry, and for which treatment methods have not been established because of low evidence.

In the present specification, the term “subject” includes mammalians such as primates including humans, pet animals such as dogs and cats, and ornamental animals such as zoo animals. Preferred subjects are humans.

In the present specification, “hydrogen,” the active ingredient of the composition of the present invention, is molecular hydrogen (i.e., gaseous hydrogen or hydrogen gas) and is simply referred to as “hydrogen” or “hydrogen gas” unless otherwise specified. Additionally, the term “hydrogen” used in the present specification refers to a molecular formula of H₂, D₂ (deuterium), or HD (deuterated hydrogen) or a gas mixture thereof. D₂ is expensive but known to have a stronger superoxide eliminating effect than that of H₂. Hydrogen that can be used in the present invention is H₂, D₂ (deuterium), HD (deuterated hydrogen), or a gas mixture thereof, preferably H₂. Alternatively, D₂, and/or HD can be used instead of H₂ or in a mixture with H₂.

Preferred embodiments of the composition of the present invention are gases or liquids containing molecular hydrogen, preferably gases containing molecular hydrogen.

The gases containing molecular hydrogen are preferably air containing hydrogen gas or a mixed gas containing hydrogen gas and oxygen gas. The concentration of hydrogen gas in a gas containing molecular hydrogen (i.e., the composition of the present invention) is higher than zero (0) and not higher than 18.5% by volume, for example, 0.5% to 18.5% by volume, preferably 1% to 10% by volume, for example, 2% to 10% by volume, 2% to 9% by volume, 2% to 8% by volume, 3% to 10% by volume, 3% to 9% by volume, 3% to 8% by volume, 3% to 7% by volume, 3% to 6% by volume, 4% to 10% by volume, 4% to 9% by volume, 4% to 8% by volume, 4% to 7% by volume, 4% to 6% by volume, 4% to 5% by volume, 5% to 10% by volume, 5% to 9% by volume, 5% to 8% by volume, 6% to 10% by volume, 6% to 9% by volume, 6% to 8% by volume, 6% to 7% by volume, and the like. In the present invention, higher hydrogen gas concentrations (but below the explosion limit) or higher daily hydrogen doses tend to be associated with greater effects of promoting prevention and/or improvement (e.g., suppression or alleviation) of pneumonia.

Because hydrogen is a flammable and explosive gas, it is preferable to add hydrogen to the composition of the present invention under conditions safe for subjects such as humans and administer the mixture to subjects to prevent and/or improve pneumonia.

When a gas other than hydrogen gas is air, the air concentration is in the range of, for example, 81.5% to 99.5% by volume.

When a gas other than hydrogen gas is a gas containing oxygen gas, the oxygen gas concentration is in the rage of, for example, 21% to 99.5% by volume.

As another main gas, for example, nitrogen gas can be further added.

In a usual hydrogen gas inhalation therapy, an effect of improving a disease (cancer) is observed only when a hydrogen gas is used at a high concentration of 66% or 99%. In the present invention, however, it is preferable to add hydrogen to the composition of the present invention under safe conditions for subjects such as humans and administer it to a subject, and a sufficient effect of improving pneumonia can be exhibited even at low hydrogen concentrations of higher than 0 (zero) and 18.5% or lower.

The liquids containing molecular hydrogen are specifically aqueous liquids containing a dissolved hydrogen gas. Examples of the aqueous liquids used herein include, but are not limited to, water (e.g., purified water, sterilized water), physiological saline, buffer solutions (e.g., buffer solutions of pH 4 to 7.4), drip infusion solutions, fluid infusion solutions, injection solutions, and drinks (e.g., tea drinks such as green tea and black tea, fruit juice, green juice, vegetable juice). Examples of the hydrogen concentration in a liquid containing molecular hydrogen include, but are not limited to, 1 to 10 ppm, preferably 1.2 to 9 ppm, for example, 1.5 to 9 ppm, 1.5 to 8 ppm, 1.5 to 7 ppm, 1.5 to 6 ppm, 1.5 to 5 ppm, 1.5 to 4 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 4 to 7 ppm, 5 to 10 ppm, 5 to 9 ppm, 5 to 8 ppm, and 5 to 7 ppm.

In the present invention, higher concentrations of dissolved hydrogen (but below the explosion limit) or higher daily hydrogen doses tend to be associated with greater effects of preventing and/or improving pneumonia.

A gas or a liquid containing molecular hydrogen is formulated to provide a predetermined hydrogen gas concentration and then with the same, for example, a pressure-resistant container (e.g., a stainless cylinder, an aluminum can, a pressure-resistant plastic bottle [e.g., a pressure-resistant PET bottle] and a plastic bag preferably having the inside laminated with an aluminum film, or an aluminum bag) is filled. Aluminum has the property of unlikely allowing hydrogen molecules to pass therethrough. Alternatively, a gas containing molecular hydrogen or a liquid containing molecular hydrogen may be produced in situ before use by using an apparatus such as a hydrogen gas generating apparatus, a hydrogen water generating apparatus, or a hydrogen gas adding apparatus such as a known or commercially available hydrogen gas supply apparatus (an apparatus for generating a gas containing molecular hydrogen), a hydrogen adding device (an apparatus for hydrogen water generation), or a non-destructive hydrogen adding apparatus (e.g., an apparatus for non-destructively adding hydrogen gas into a bag for a biocompatible solution such as a drip infusion solution).

The hydrogen gas supply apparatus enables hydrogen gas generated from a reaction of a hydrogen generating agent (e.g., metallic aluminum, magnesium hydride) and water to be mixed with a diluent gas (e.g., air, oxygen) in a predetermined ratio (refer to Japanese Patent No. 5228142, etc.). Or, the hydrogen gas supply apparatus mixes hydrogen gas generated utilizing electrolysis of water with a diluent gas such as oxygen or air (refer to Japanese Patent No. 5502973, Japanese Patent No. 5900688, etc.). Thus, a gas containing molecular hydrogen at a hydrogen concentration in the range of, for example, 0.5% to 18.5% by volume can be prepared.

The hydrogen adding device is an apparatus that generates hydrogen by using a hydrogen generating agent and a pH modifier and dissolving the hydrogen in a biocompatible solution such as water (refer to Japanese Patent No. 4756102, Japanese Patent No. 4652479, Japanese Patent No. 4950352, Japanese Patent No. 6159462, Japanese Patent No. 6170605, Japanese Patent Laid-open No. 2017-104842, etc.). Examples of a mixture of a hydrogen generating agent and a pH modifier include metallic magnesium and a strongly acidic ion exchange resin or an organic acid (e.g., malic acid, citric acid) and a metallic aluminum powder and a calcium hydroxide powder. With these mixtures, a liquid containing molecular hydrogen at a dissolved hydrogen concentration of, for example, approximately 1 to 10 ppm can be prepared.

The non-destructive hydrogen adding apparatus is an apparatus or a device that adds hydrogen gas to a commercially available biocompatible solution such as a drip infusion solution (e.g., enclosed in a hydrogen-permeable plastic bag such as a polyethylene bag) from the outside of a package and is commercially available from, for example, MiZ Company Limited (http://www.e-miz.co.jp/technology.html). This apparatus can dissolve hydrogen in a biocompatible solution aseptically until the equilibrium concentration is reached, by immersing a bag containing the biocompatible solution in saturated hydrogen water, so that hydrogen is permeated into the bag. The apparatus is composed of, for example, an electrolytic bath and a water bath, and water in the water bath is circulated in the electrolytic bath and the water bath to generate hydrogen by electrolysis. Or, a simplified, disposable device can be used for a similar purpose (refer to Japanese Patent Laid-open No. 2016-112562, etc.). This device has a biocompatible solution-containing plastic bag (a hydrogen-permeable bag, for example, a polyethylene bag) and a hydrogen generating agent (e.g., metallic calcium, metallic magnesium/cation exchange resin) incorporated in an aluminum bag, and the hydrogen generating agent is wrapped with, for example, a non-woven fabric (e.g., steam-permeable non-woven fabric). Hydrogen generated by wetting the hydrogen generating agent wrapped with a non-woven fabric with a small amount of water, such as a steam, is dissolved in a biocompatible solution non-destructively and aseptically.

Or, a purified hydrogen gas cylinder, a purified oxygen gas cylinder, or a purified air cylinder may be provided to produce a gas or a liquid containing molecular hydrogen which is adjusted to provide a predetermined hydrogen concentration or a predetermined oxygen or air concentration.

A gas containing molecular hydrogen or a liquid containing molecular hydrogen (e.g., water [e.g., purified water, sterilized water], physiological saline, drip infusion solution) prepared using the above-mentioned apparatuses or devices can be administered orally or parenterally to subjects with pneumonia.

Other embodiments of the composition of the present invention include dosage forms (e.g., tablets, capsules) prepared to be orally administered to (or ingested by) subjects, which contain a hydrogen generating agent that enables hydrogen to be generated in the gastrointestinal tract. The hydrogen generating agent preferably comprises, for example, components approved as food or food additives.

When the composition of the present invention comprises molecular hydrogen as an active ingredient, examples of the method of administering the composition to subjects include administration by inhalation, suction or the like. For example, transpulmonary administration is preferred. When a liquid containing molecular hydrogen is contained as an active ingredient, oral or intravenous administration (including drip infusion) is preferred. When a gas is inhaled, the gas is inhaled from the mouth or the nose via a nasal cannula or a mask-like device covering the mouth and the nose, transported to the lungs, and delivered to the whole body by blood.

The liquid containing molecular hydrogen to be orally administered may be administered to subjects as a cooled liquid or a liquid stored at room temperature. Hydrogen is dissolved in water at a concentration of approximately 1.6 ppm (1.6 mg/L) at room temperature and under a normal pressure, and the difference in solubility due to temperature is known to be relatively small. Or, when a liquid containing molecular hydrogen is, for example, in the form of a drip infusion solution or an injection solution containing hydrogen gas prepared using the above-described non-destructive hydrogen adding apparatus, the liquid may be administered to subjects by parenteral routes, such as intravenous or intraarterial administration.

One dose or multiple doses (e.g., two to three doses) per day of a gas containing molecular hydrogen at the above-mentioned hydrogen concentrations or a liquid containing molecular hydrogen at the above-mentioned dissolved hydrogen concentrations can be administered to humans for a period of one week to three months or longer, for example, one week to six months or longer (e.g., one year or longer, two years or longer). When a gas containing molecular hydrogen is administered, the gas is preferably inhaled for at least 30 minutes per dose. Because the improving effect becomes higher with a longer inhalation time, the gas can be administered for, for example, 30 minutes to one hour, one hour to two hours, two hours to three hours, or longer. Additionally, when a gas containing molecular hydrogen is administered in a transpulmonary manner by inhalation or suction, the gas can be administered to subjects under an atmospheric pressure environment, or, for example, under a high atmospheric pressure in the range exceeding a standard atmospheric pressure (i.e., approximately 1.013 atm) and not higher than 7.0 atm, for example, under a high atmospheric pressure environment in the range of 1.02 to 7.0 atm, preferably in the range of 1.02 to 5.0 atm, more preferably in the range of 1.02 to 4.0 atm, yet more preferably in the range of 1.02 to 1.35 atm (including the gas containing molecular hydrogen).

2. A Method for Preventing and/or Improving Pneumonia

The composition containing molecular hydrogen, pneumonia, symptom associated with pneumonia, dose, administration method, and the like are as described in the above 1.

In the method of the present invention, a gas containing molecular hydrogen (preferably, air or oxygen) at higher than zero (0) and not higher than 18.5% by volume, for example, 0.5% to 18.5% by volume, 2% to 10% by volume, 2% to 9% by volume, 2% to 8% by volume, 3% to 10% by volume, 3% to 9% by volume, 3% to 8% by volume, 3% to 7% by volume, 3% to 6% by volume, 4% to 10% by volume, 4% to 9% by volume, 4% to 8% by volume, 4% to 7% by volume, 4% to 6% by volume, 4% to 5% by volume, 5% to 10% by volume, 5% to 9% by volume, 5% to 8% by volume, 6% to 10% by volume, 6% to 9% by volume, 6% to 8% by volume, 6% to 7% by volume, or the like, preferably 5% to 10% by volume, 5% to 8% by volume, for example, 6% to 10% by volume, 6% to 8% by volume, 6% to 7% by volume, or the like can be inhaled or sucked by subjects for, for example, one to three hours or longer per day and can be continued for, for example, one to three months or longer, four to seven months or longer, one to three years or longer.

Or, in the method of the present invention, for example, 200 to 500 ml per dose for intravenous administration or, for example, 500 to 1000 ml per dose for oral administration of a liquid containing molecular hydrogen at a concentration of, for example, 1 to 10 ppm, 1.5 to 9 ppm, 1.5 to 8 ppm, 1.5 to 7 ppm, 1.5 to 6 ppm, 1.5 to 5 ppm, 1.5 to 4 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 4 to 7 ppm, 5 to 10 ppm, 5 to 9 ppm, 5 to 8 ppm, 5 to 7 ppm, or the like, preferably 3 to 10 ppm, 4 to 10 ppm, 5 to 10 ppm, 5 to 9 ppm, 5 to 8 ppm, 5 to 7 ppm, or the like can continue to be administered to subjects for, for example, 0.5 to three months or longer, four to seven months or longer, one to three years or longer.

The method of the present invention may further be used in combination with a therapeutic agent used for the treatment of pneumonia, if necessary. Such a combination use is expected to increase levels of prevention and/or improvement of pneumonia.

### EXAMPLE

The present invention is explained more specifically with reference to the following example. However, the example is not intended to limit the scope of the present invention.

### Example 1

### <A Case of Interstitial Pneumonia Improved by Hydrogen Inhalation>

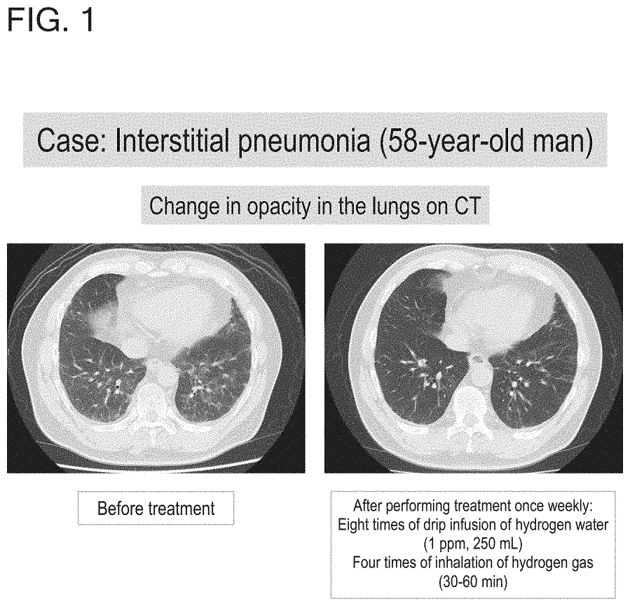

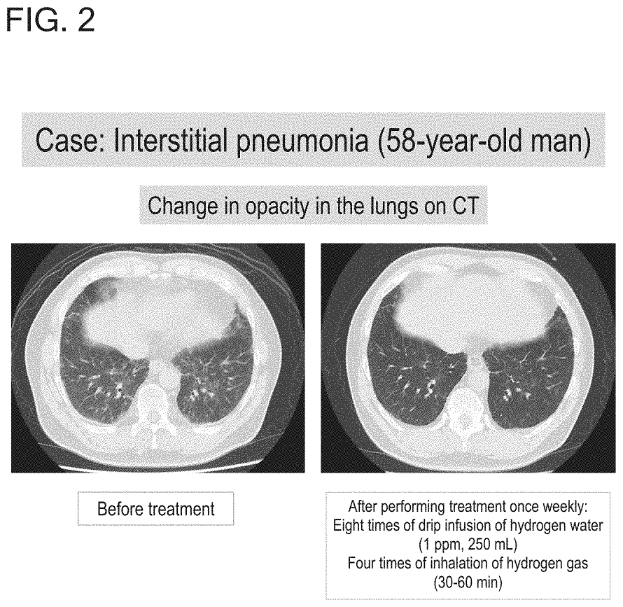

A 58-year-old male patient who was diagnosed as having interstitial pneumonia received a drip infusion of hydrogen (1 ppm hydrogen, 250 ml) and inhalation of hydrogen gas over 30 minutes to 60 minutes once weekly for 8 weeks. MHG-2000α manufactured by MiZ Company Limited (hydrogen gas concentration 6 to 7%, 120 ml/min) was used for inhalation of a hydrogen gas. Significant improvement of interstitial pneumonia by administration of the hydrogen was confirmed by disappearance of ground-glass opacity in the CT finding. CT photographs taken before and after treatment are shown in FIG. 1 and FIG. 2.

The present invention can prevent and/or improve pneumonia by administering a composition containing molecular hydrogen.

What is claimed is:

1. A method for preventing and/or improving pneumonia and/or a symptom associated with pneumonia in a subject, comprising administering to the subject a composition comprising an effective amount of molecular hydrogen.

2. The method according to claim 1, wherein the pneumonia is one or more pneumonia selected from the group consisting of bacterial pneumonia, viral pneumonia, fungal pneumonia, radiation pneumonia, and drug-induced pneumonia.

3. The method according to claim 1, wherein the symptom associated with pneumonia is one or more symptoms selected from the group consisting of sore throat, pyrexia, coughing, sneezing, sputum production, dyspnea, systemic malaise, nausea, fatigue, chest pain, shortness of breath, rhinorrhea, decreases in white blood cells, diarrhea, headache, reduced lung function, reduced heart function, and reduced kidney function.

4. The method according to claim 1, wherein the composition is a liquid composition or a gas composition comprising the molecular hydrogen.

5. The method according to claim 4, wherein the liquid composition has a hydrogen concentration of 1 to 10 ppm.

6. The method according to claim 4, wherein the gas composition has a hydrogen concentration of higher than zero (0) and not higher than 18.5% by volume.

7. The method according to claim 1, wherein the subject is a mammalian subject.

8. The method according to claim 1, wherein the subject is a human subject.

9. The method according to claim 1, wherein the composition is produced by using a hydrogen gas generating apparatus, a hydrogen water generating apparatus, or a hydrogen gas adding apparatus.
